(19) United States
(12) Patent Application Publication
(10) Pub. No.: US 2011/0172512 A1
(43) Pub. Date: Jul. 14, 2011

(54) METHOD AND SYSTEM FOR MAGNETIC INDUCTION TOMOGRAPHY

(75) Inventors: Ming Yan, Shanghai (CN); Dayu Chen, Shanghai (CN); Hua Jin, Shanghai (CN)

(73) Assignee: KONINKLIJKE PHILIPS ELECTRONICS N.V., EINDHOVEN (NL)

(21) Appl. No.: 13/063,033

(22) PCT Filed: Sep. 2, 2009
(86) PCT No.: PCT/IB09/53817

§ 371 Date: Mar. 9, 2011

(30) Foreign Application Priority Data
Sep. 11, 2008 (CN) 200810215357.6

(51) Int. Cl. A61B 5/05

(52) U.S. Cl. 600/407

(57) ABSTRACT

The invention relates to a method and a system for magnetic induction tomography, the system comprising: at least one transmitting coil for generating a primary magnetic field to be applied to the object of interest; and at least one measurement coil arrangement for measuring electric signals induced by a secondary magnetic field which is generated by the object of interest in response to the primary magnetic field, wherein the at least one measurement coil arrangement comprises a plurality of measurement coils which are positioned in substantially the same plane. By using a plurality of independent measurement coils positioned in a plane so as to replace a conventional single measurement coil, the measurement coil across which the measured difference voltage is most sensitive to a change of the secondary magnetic field can be selected for calculating the change of conductivity distribution, resulting in an improved sensitivity of a MIT system.

### FIELD OF THE INVENTION

The invention relates to magnetic induction tomography, particularly to a method and system for improving the sensibility of a magnetic induction tomography system.

### BACKGROUND OF THE INVENTION

Magnetic induction tomography (MIT) is a non-invasive and contactless imaging technique with applications in industry and medical imaging. In contrast to other electrical imaging techniques, MIT does not require direct contact of the sensors with the object of interest for imaging.

MIT is used to reconstruct the spatial distribution of the passive electrical properties inside the object of interest, for example, conductivity σ, permittivity ε and permeability μ. In MIT, a sinusoidal electric current, normally between a few kHz up to several MHz, is applied to a transmitting coil generating a time-varying magnetic field, usually referred to as primary magnetic field. Due to the conducting object of interest, for example, a biological tissue, the primary field produces “eddy currents” in the object of interest. These eddy currents generate a secondary magnetic field. The combination of these magnetic fields induces an electric signal, for example, electric voltages in the receiving coils. Using several transmitting coils and repeating the measurements, sets of measurement data are acquired and used to visualize changes in time of the electromagnetic properties of the object of interest.

MIT is sensitive to all three passive electromagnetic properties: electrical conductivity, permittivity and magnetic permeability. As a result, for example, the conductivity contribution in the object of interest can be reconstructed. In particular, MIT is suitable for reconstructing images for biological tissue, because of the magnetic permeability value of such tissue μ_{R}≈1.

The secondary magnetic field induced by the eddy current carries information about the object to be measured. However, the voltage ΔV induced by the secondary magnetic field is very small and the ratio of the voltage ΔV to the measured voltage V on the measurement coil, e.g. |ΔV/V| can be as small as 10⁻⁷ on some coils. This introduces at least two problems in current MIT systems: first, the system requires high-precision ADC, which increases the hardware cost, and secondly, the system is very sensitive to noise and thus imposes limitations on its detection performance.

Prior-art document “A new type of gradiometer for the receiving circuit of magnetic induction tomography (MIT)”, by Hermann etc. in Physiol. Meas, Vol. 26, pp. S307-S318, 2005, discloses a method of subtracting signals in a pair of differential coils by using a gradiometer for the receiving circuit, which improves the sensitivity of a MIT system.

However, gradiometer coils are very sensitive to the geometry in symmetry in coil arrangements. When the coil pair deviates from the ideal symmetrical shape, for example, because of a deformation caused by mechanical and/or temperature instability, the coils do not compensate each other perfectly.

### SUMMARY OF THE INVENTION

It would be advantageous to achieve an image reconstruction system having improved sensitivity of the system.

To better address one or more of these concerns, in a first aspect of the invention, a system for reconstructing images of an object of interest is provided, and the system comprising:
at least one transmitting coil configured to generate a primary magnetic field to be applied to the object of interest; and
at least one measurement coil arrangement configured to measure electric signals induced by a secondary magnetic field which is generated by the object of interest in response to the primary magnetic field;
   wherein the at least one measurement coil arrangement comprises a plurality of measurement coils which are positioned in substantially the same plane.

By using a plurality of independent measurement coils positioned in a plane so as to replace a conventional single measurement coil, the change of the secondary magnetic field caused by a change of conductivity distribution of the object of interest can be calculated independently, resulting in an improved sensitivity when used in, for example, a MIT system.

It is advantageous that the system further comprises a processor configured to reconstruct images of said object of interest, based on the measured electric signals induced by the secondary magnetic field, the processor having a control unit for controlling each of the plurality of measurement coils to measure a first and a second electric signal thereon.

In one embodiment, the first and the second electric signal are induced voltages, and the processor further comprises a first selecting unit configured to select a measurement coil from the plurality of measurement coils, the selected measurement coil having the largest absolute value of the ratio of the difference voltage between the first and the second voltage to the first voltage across said coil.

In another embodiment, the first and the second electric signals are induced voltages, and the processor further comprises a second selecting unit configured to select a measurement coil from the plurality of measurement coils, the selected measurement coil having the largest absolute value of the difference voltage between the first and the second voltage across said coil.

It is advantageous that the processor further comprises a first calculator configured to calculate the change of conductivity distribution of the object of interest based on the difference voltage corresponding to the selected measurement coil.

The invention improves the sensitivity of the MIT system by selecting the measurement coil which is most sensitive to the change of the secondary magnetic field, and by using the difference voltage corresponding to the selected measurement coil in image reconstruction.

It is also advantageous that the processor further comprises a second calculator configured to calculate the change of conductivity distribution of the object of interest based on a plurality of weighted difference voltages derived from the plurality of first and second voltages.

By weighting the difference voltages generated by the plurality of measurement coils, more independent measurement data can be used for image reconstruction, resulting in an improved sensitivity of the MIT system.

According to another aspect of the invention, it provides a method of reconstructing images of an object of interest, said method comprising the steps of:
(a) generating a primary magnetic field to be applied to the object of interest by at least one transmitting coil; and
(b) measuring electric signals induced by a secondary magnetic field by at least one measurement coil arrangement comprising a plurality of measurement coils which are positioned in substantially the same plane, the secondary magnetic field being generated by the object of interest in response to the primary magnetic field.

Detailed explanations and other aspects of the invention are given below.

### BRIEF DESCRIPTION OF THE DRAWINGS

The above and other objects and features of the present invention will become more apparent from the following detailed description with reference to the accompanying drawings, in which:

FIG. 1 is a schematic view of a conventional measurement coil arrangement.

FIG. 2 is a schematic view of an embodiment of a measurement coil arrangement according to the invention.

FIG. 3 is a schematic view of an embodiment of a system according to the invention.

FIG. 4 is a schematic flowchart of the method according to the invention.

The same reference numerals are used to denote similar parts throughout the Figures.

### DESCRIPTION OF EMBODIMENTS

In a MIT system, the relationship between the object conductivity distribution and measured voltages on coils is set up with Maxwell equations. The voltage across the measurement coil is the integration of electric field (E) over the coil:

$$V = {∮}_{C} E d \vec{l} \qquad ( 1 )$$

In the Maxwell equation, the relationship between the conductivity and the E field is derived as follows:

∇×(μᵣ⁻¹∇×E)−εᵣκ²E=iκμ₀^{1/2}Ĵₐ (2)

In this equation, Ĵₐ is the applied current density on transmitting coils, and

$${μ}_{r} = \frac{μ}{{μ}_{0}} and {ɛ}_{r} = \frac{ɛ}{{ɛ}_{0}} + \frac{i σ}{{ɛ}_{0} ω} \qquad ( 3 )$$

wherein σ, μ and ε are the conductivity, permeability and permittivity, respectively, and μ₀ and ε₀ are the permittivity and permeability of the free space and κ=ω\sqrt{ε₀μ₀}, respectively. The E field at any point in the space cannot be measured directly, and instead, the voltage V which is the integration of the E field along the coil is measured.

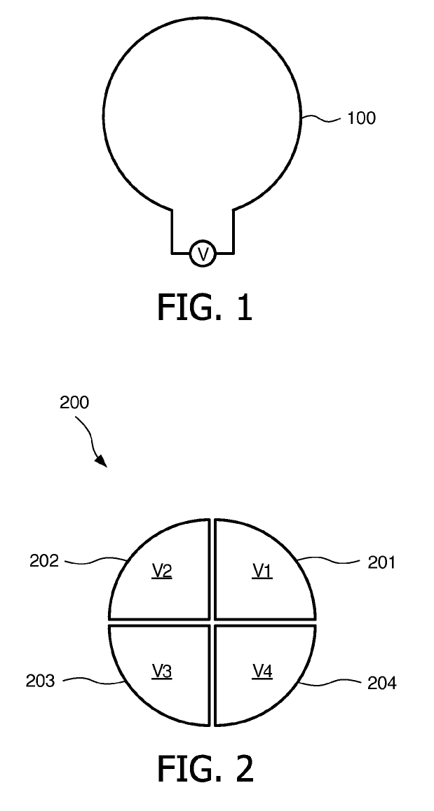

FIG. 1 is a schematic view of a measurement coil arrangement comprising a single measurement coil 100 to be used in a conventional MIT system. As the E field is a 3D vector field in the space, and if the coil is divided into four segments, the measurements on the four segments are different.

For the same reason, the voltage difference ΔV on four segments, corresponding to the change of conductivity distribution, will be different. The voltage difference on one of the four segments of the coil will be most sensitive to a change of conductivity distribution of the object of interest.

However, it is difficult to measure the voltage across each coil segment. This means that the coil can only measure the voltage induced by the change of a secondary magnetic field through the area enclosed by the coil and cannot identify which coil segment is most sensitive to the change of the secondary magnetic field caused by a change of conductivity of an object of interest.

Based on the understanding and insight of the relationship between the measured voltages across coils and the change of conductivity distribution, the invention provides a system comprising a novel measurement coil arrangement for measuring the voltage that is most sensitive to a change of conductivity distribution by using a plurality of independent measurement coils positioned in substantially the same plane so as to replace a conventional single measurement coil.

FIG. 2 is a schematic view of an embodiment of a measurement coil arrangement 200 according to the invention.

The measurement coil arrangement comprises a plurality of independent coils which are positioned in substantially the same plane. In this embodiment, the measurement coil arrangement comprises four independent coils 201, 202, 203 and 204.

The coils may have different shapes, for example, they may be fan-shaped or square-shaped. Accordingly, the areas enclosed by the coils may be substantially the same or different so as to adapt to different applications.

It is advantageous that the coils are printed on a printed circuit board (PCB) and a sampling channel is used to read measurement data from each measurement coil.

By using a plurality of independent measurement coils positioned in a plane so as to replace a conventional single measurement coil, the change of the secondary magnetic field caused by a change of conductivity distribution of the object of interest can be calculated independently.

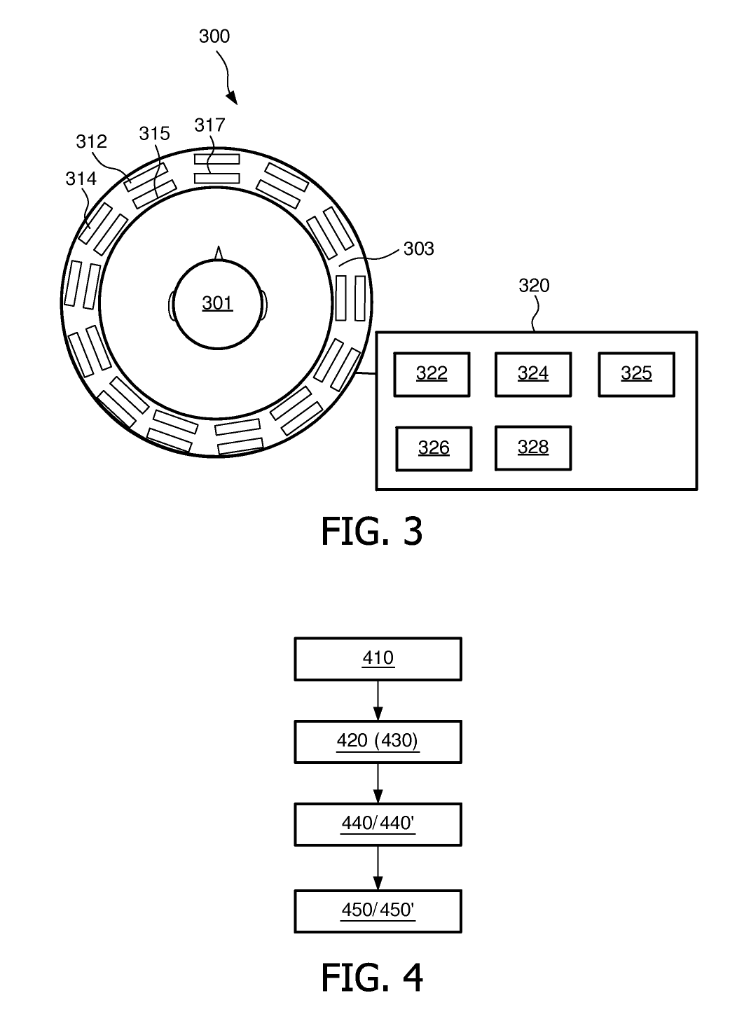

FIG. 3 is a schematic view of an embodiment of a system 300 according to the invention.

The system 300 comprises transmitting coils 312, 314 configured to generate a primary magnetic field. The primary magnetic field induces an eddy current in an object of interest 301. The object of interest 301 may be the head of a human being or a block of conductive material. For example, the transmitting coils 312, 314 are supplied by an alternating current so as to generate the primary magnetic field.

The system 300 further comprises at least one measurement coil arrangement 315, 317 configured to measure electric signals induced by a secondary magnetic field which is generated by the object of interest in response to the primary magnetic field. In particular, the secondary magnetic field is generated by the eddy current in the object of interest which is induced by the primary magnetic field. Each measurement coil arrangement comprises a plurality of measurement coils which are positioned in a plane as shown in FIG. 2. The transmitting coils 312, 314 and the measurement coil arrangement 315, 317 may be arranged on a rack 303.

The system 300 further comprises a processor 320 configured to reconstruct images based on the measured electric signals, for example, the induced voltages across the coils. The detailed implementation of the processor 320 will be explained hereinafter with reference to FIG. 2 and FIG. 3.

As shown in FIG. 3, the processor comprises a control unit 322 configured to control each of the plurality of measurement coils so as to measure a first and a second electric signal thereon before and after a change of conductivity distribution of the object of interest.

This means that each coil measures a first voltage and a second electric signal, for example, the voltage induced on the measurement coil. The difference voltage between the first and the second voltage results from the change of conductivity distribution of the object of interest.

In case the measurement coil has four measurement coils as shown in FIG. 2, there are four groups of measurements: Vᵢ¹ and Vᵢ², i=1, 2, 3, 4, Vᵢ¹ and Vᵢ² denote the voltage measured before and after the change of conductivity distribution. Usually Vᵢ¹ and Vᵢ² are the respective sums of the voltage induced by the primary and secondary magnetic fields.

It should be noted that the measuring control may be sequential, i.e. each coil measures one after the other, or in parallel, i.e. all coils measure at the same time, in dependence upon the configuration of hardware for collecting measurement data.

There are different ways of selecting/identifying a measurement coil on which the induced voltage is more sensitive to the change of conductivity distribution. The sensitivity of the measured voltage to a change of conductivity distribution for each measurement can be defined as:

$${S}_{i} = \vert \frac{Δ {V}_{1}}{{V}_{i}^{1}} \vert \qquad ( 4 )$$

wherein ΔV=Vᵢ²−Vᵢ¹ denotes the voltage change corresponding to the change of conductivity distribution.

As the voltage ΔVᵢ generated by the change of the secondary magnetic field is very small as compared to the measured voltage Vᵢ¹ or Vᵢ², the sensitivity Sᵢ can be as small as 10⁻⁷. When the shape and the area enclosed by each coil is substantially the same, the difference between Vᵢ¹ for different coils may be very limited, and in such a situation, ΔVᵢ can be used for indicating the sensitivity.

In one embodiment, the processor further comprises a first selecting unit 324 configured to select a measurement coil from the plurality of measurement coils, the selected measurement coil having the largest absolute value of the ratio of the difference voltage between the first and the second voltage to the first voltage across said coil. This means that the measurement coil having the largest absolute value of Sᵢ is selected.

In another embodiment, the processor further comprises a second selecting unit 325 configured to select a measurement coil from the plurality of measurement coils, the selected measurement coil having the largest absolute value of the difference voltage between the first and the second voltage across said coil. This means that the measurement coil having the largest absolute value of ΔVᵢ is selected.

In a further embodiment, the processor also comprises a first calculator 326 configured to calculate the change of conductivity distribution of the object of interest based on the difference voltage ΔVᵢ corresponding to the selected measurement coil.

The calculation of the change of conductivity distribution of the object of interest may follow known image reconstruction theories, for example, the method of conductivity calculations and image reconstruction described in the prior-art document “Image reconstruction approaches for Philips magnetic induction tomograph”, M. Vauhkonen, M. Hamsch and C. H. Igney, ICEBI 2007, IFMBE Proceedings 17, pp. 468-471, 2007.

The set of parameters can be calculated in accordance with the following equation, e.g. equation (8) in the mentioned prior art:

Δσ=(J^{T}W^{T}WJ+αL^{T}L)⁻¹(J^{T}W^{T}WΔVᵢ) (5)

wherein W is a weighting matrix, α is a regularization parameter and L is a regularization matrix, J is the imaginary part of the complex Jacobian matrix, ΔVᵢ is the difference voltage between Vᵢ¹ and Vᵢ² and corresponds to the change of conductivity distribution.

In another embodiment, the processor further comprises a second calculator 328 configured to calculate the change of conductivity distribution of the object of interest based on a plurality of weighted difference voltages derived from the plurality of first and second voltages. In the calculation of the change of conductivity distribution,

$${\sum }_{i = 1}^{M} {w}_{i} \cdot Δ {V}_{i}$$

is used to replace ΔVᵢ in Equ. (5), wherein M is the number of measurement coils in a measurement coil arrangement, for example, M=4 when using the measurement coil arrangement shown in FIG. 2. In this way, all measurements obtained by the plurality of measurement coils contribute to the calculation of the change of conductivity distribution. The weighting parameter wᵢ can be computed with this method.

$${w}_{i} = \frac{Δ {V}_{i}}{{\sum }_{j = 1}^{4} Δ {V}_{j}} \qquad ( 6 )$$

The Jacobian Matrix is also weighted with the w, so as to get the new Jacobian matrix for reconstruction.

FIG. 4 is a schematic flowchart of the method according to the invention.

According to the invention, the method comprises a step 410 of generating a primary magnetic field using at least one or more transmitting coils 312, 314, the primary magnetic field inducing an eddy current in an object of interest 301.

The method further comprises a step 420 of measuring signals induced by a secondary magnetic field generated by the eddy current for image reconstruction by using at least one measurement coil arrangement 315, 317 comprising a plurality of measurement coils 201, 202, 203, 204 which are positioned in a plane 200.

It is advantageous that the method further comprises a step 430 of controlling each of the plurality of measurement coils so as to measure a first and a second voltage Vᵢ¹, Vᵢ² before and after a change of conductivity distribution of the object of interest for image reconstruction.

In one embodiment, the method further comprises a step 440 of selecting a measurement coil from the plurality of measurement coils, the selected measurement coil having the largest absolute value of the ratio of the difference voltage between the first and the second voltage to the first voltage.

In another embodiment, the method further comprises a step 440′ of selecting a measurement coil from the plurality of measurement coils, the selected measurement coil having the largest absolute value of the difference voltage between the first and the second voltage. Step 440′ can be executed to replace step 440.

The method further comprises a step 450 of calculating the change of conductivity distribution of the object of interest based on the difference voltage corresponding to the selected measurement coil, using Equ. (5).

In another embodiment, the method further comprises a step 450′ of calculating the change of conductivity distribution of the object of interest based on a plurality of weighted difference voltages derived from the plurality of first and second voltages. In such a situation,

$${\sum }_{i = 1}^{M} {w}_{i} \cdot Δ {V}_{i}$$

is used to replace ΔVᵢ in Equ. (5) for calculating the change of conductivity distribution, i.e. step 450′ is executed to replace step 450, and the method goes directly from step 430 to step 450′.

It should be noted that the selection of the measurement coil and/or calculation of the change of conductivity distribution can be advantageously implemented by computer programs, and/or in combination with hardware and software.

It should also be noted that the above-mentioned embodiments illustrate rather than limit the invention and that those skilled in the art will be able to design alternative embodiments without departing from the scope of the appended claims. In the claims, any reference signs placed between parentheses shall not be construed as limiting the claim. Use of the verb “comprise” and its conjugations does not exclude the presence of elements or steps other than those stated in a claim or in the description. Use of the indefinite article “a” or “an” preceding an element does not exclude the presence of a plurality of such elements. The invention can be implemented by means of hardware comprising several distinct elements and a programmed computer. In the system claims enumerating several means, several of these means can be embodied by one and the same item of hardware or software. Use of the words first, second and third, etc. does not indicate any ordering. These words are to be interpreted as names.

1. A system (300) for reconstructing images of an object of interest (301), said system comprising:
at least one transmitting coil (312, 314) configured to generate a primary magnetic field to be applied to the object of interest; and
at least one measurement coil arrangement (315, 317) configured to measure electric signals induced by a secondary magnetic field which is generated by the object of interest in response to the primary magnetic field;
wherein the at least one measurement coil arrangement (315, 317) comprises a plurality of measurement coils (201, 202, 203, 204) which are positioned in substantially the same plane.

2. A system as claimed in claim 1 further comprising a processor (320) for reconstructing images of said object of interest, based on the measured electric signals induced by the secondary magnetic field, the processor having a control unit (322) configured to control each of the plurality of measurement coils to measure a first and a second electric signal thereon.

3. A system as claimed in claim 2, wherein the first and the second electric signal are induced voltages, and the processor further comprises a first selecting unit (324) configured to select a measurement coil from the plurality of measurement coils, the selected measurement coil having the largest absolute value of the ratio of the difference voltage between the first and the second voltage to the first voltage across said coil.

4. A system as claimed in claim 2, wherein the first and the second electric signals are induced voltages, and the processor further comprises a second selecting unit (325) configured to select a measurement coil from the plurality of measurement coils, the selected measurement coil having the largest absolute value of the difference voltage between the first and the second voltage across said coil.

5. A system as claimed in claim 3, wherein the processor further comprises a first calculator (326) configured to calculate the change of conductivity distribution of the object of interest based on the difference voltage corresponding to the selected measurement coil.

6. A system as claim in claim 2, wherein the processor further comprises a second calculator (328) configured to calculate the change of conductivity distribution of the object of interest based on a plurality of weighted difference voltages derived from the plurality of first and second voltages.

7. A system as claimed in claim 1, wherein each of the plurality of measurement coils is fan-shaped, and the plurality of measurement coils forms a circular plane.

8. A system as claimed in claim 1, wherein each of the plurality of measurement coils is square-shaped, and the plurality of measurement coils forms a square plane.

9. A system as claimed in claim 7, wherein each of the plurality of measurement coils encloses substantially the same area.

10. A magnetic induction tomography scanner comprising a system as claimed in claim 1.

11. A method of reconstructing images of an object of interest, said method comprising the steps of:
(a) generating (410) a primary magnetic field to be applied to the object of interest by at least one transmitting coil; and
(b) measuring (420) electric signals induced by a secondary magnetic field by at least one measurement coil arrangement comprising a plurality of measurement cols which are positioned in substantially the same plane, the secondary magnetic field being generated by the object of interest in response to the primary magnetic field.

12. A method as claimed in claim 11, wherein step (b) comprises a step (430) of controlling each of the plurality of measurement coils to measure a first and a second electric signal before and after a change of conductivity distribution of the object of interest.

13. A method as claimed in claim 12, wherein the first and the second electric signal are induced voltages, the method further comprising a step (440) of selecting a measurement coil from the plurality of measurement coils, the selected measurement coil having the largest absolute value of the ratio of the difference voltage between the first and the second voltage to the first voltage.

14. A method as claimed in claim 12, wherein the first and the second electric signal are induced voltages, the method further comprising a step (440′) of selecting a measurement coil from the plurality of measurement coils, the selected measurement coil having the largest absolute value of the difference voltage between the first and the second voltage.

15. A method as claimed in claim 13, further comprising a step (450) of calculating the change of conductivity distribution of the object of interest based on the difference voltage corresponding to the selected measurement coil.

16. A method as claimed in claim 12, further comprising a step (450′) of calculating the change of conductivity distribution of the object of interest based on a plurality of weighted difference voltages derived from the plurality of first and second voltages.
